(19) United States
(12) Patent Application Publication
(10) Pub. No.: US 2020/0060785 A1
(43) Pub. Date: Feb. 27, 2020

(54) SMART HANDLE FOR SURGICAL LAMP

(71) Applicant: Amico Clinical Solutions Corporation, Richmond Hill (CA)

(72) Inventors: Varun CHANDAN, Caledon (CA); Wayne BENSON, Newmarket (CA)

(21) Appl. No.: 16/547,494

(22) Filed: Aug. 21, 2019

Related U.S. Application Data
(60) Provisional application No. 62/720,581, filed on Aug. 21, 2018.

(51) Int. Cl. A61B 90/30; F21V 21/40; H05B 37/02

(52) U.S. Cl. CPC A61B 90/30; F21V 21/403; F21W 2131/205; A61B 2090/308; H05B 37/0227

(57) ABSTRACT

A surgical lamp with a handle may be equipped with a touch sensor at the end of the handle. A touch on the touch sensor may be used to toggle the lamp between on and off states. Additionally, touches on the touch sensor may be used to cycle through various brightness levels. A twist on the handle may be used to cycle through color temperature options.

### FIELD

The present application relates generally to surgical lamps and, more specifically, to a smart handle for a surgical lamp.

### BACKGROUND

Surgical lamps, like other lamps, are generally able to toggle between two states; an on state and an off state. Of course, the on state may be associated with one of a plurality of brightness levels and/or a plurality of color temperatures.

As is well known, the environment in which a surgical lamp is to be employed is an environment in which it is important that surfaces remain sterile.

### BRIEF DESCRIPTION OF THE DRAWINGS

Reference will now be made, by way of example, to the accompanying drawings which show example implementations; and in which:

FIG. 1 illustrates, in a perspective view, a lamp with a lamp head having a handle according to aspects of the present application; and

FIG. 2 illustrates, in sectional view, the handle of FIG. 1 according to aspects of the present application.

### DETAILED DESCRIPTION

It may be recognized as a challenge to toggle a surgical lamp between states or to select a brightness level or color temperature while maintaining the sterile nature of the environment.

Aspects of the present application relate to a surgical lamp with a handle that is equipped with a touch sensor at the end of the handle. A user may interact with the touch sensor to toggle the state of the lamp between on and off states.

According to an aspect of the present disclosure, there is provided a lamp. The lamp includes a lamp element, a power distribution circuit arranged to control a flow of electricity to the lamp element, a head housing the lamp element and the power distribution circuit, a handle attached, at a proximal end, to the head, the handle housing a control circuit board and a touch sensor in communication with the control circuit board, the sensor disposed at a distal end of the handle. The control circuit board is adapted to receive a signal from the touch sensor, the signal indicative of detection of a touch and responsive to the receiving, transmit an instruction to the power distribution circuit, the instruction causing the power distribution circuit to change a state of the lamp element.

Other aspects and features of the present disclosure will become apparent to those of ordinary skill in the art upon review of the following description of specific implementations of the disclosure in conjunction with the accompanying figures.

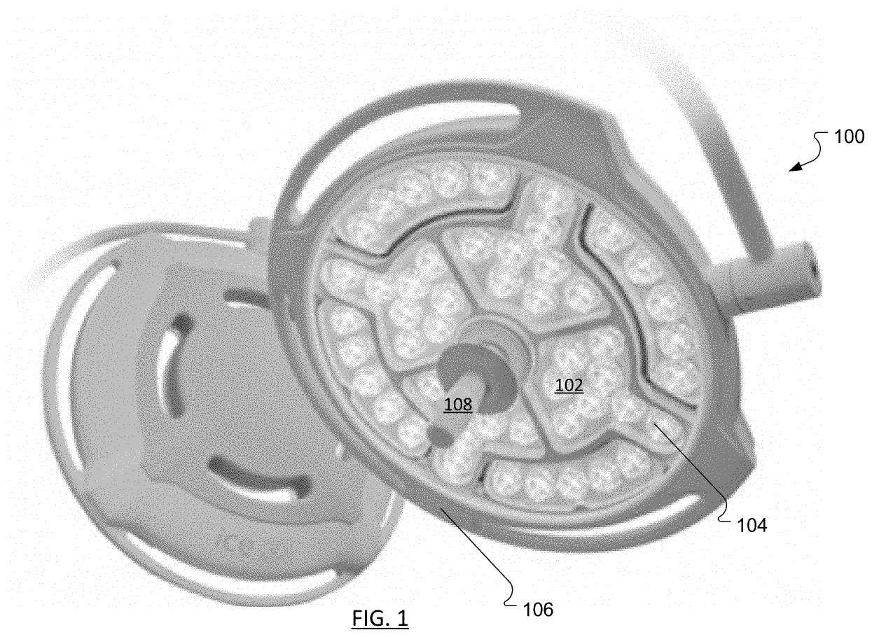

FIG. 1 illustrates a lamp 100 having a plurality of lamp element assemblies 102. Mounted on each lamp element assembly 102 of the plurality of lamp element assemblies 102 is a plurality of lamp elements 104. The lamp element assemblies 102 may be mounted within a lamp head 106. Mounted to the lamp head 106 is a handle 108. Notably, the shape of the lamp head 106 and the position of the handle 108 are design choices that, generally, do not have a bearing on the operation of the lamp 100. In FIG. 1, the lamp head 106 has a generally circular shape and the handle 108 is positioned to emanate from the center of the circular lamp head 106 in a manner that is generally normal to the flat, illuminating face of the lamp 100.

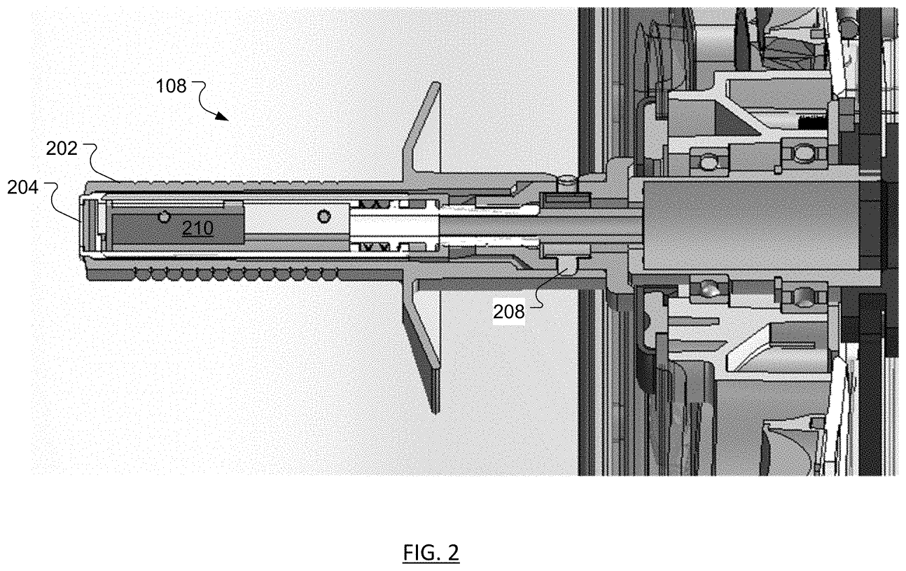

FIG. 2 illustrates, in sectional view, the handle 108 of FIG. 1. At the tip of the handle 108 is a sensor 204. The sensor 204 may, for example, be implemented as a capacitive touch sensor. The sensor 204 is connected to a control circuit board 210 disposed inside the handle 108. Power to the control circuit board 210 may be provided via a power connection (not shown) from a power distribution circuit (not shown) responsible for receiving mains power and distributing the power to the lamp elements 104 and other components of the lamp 100. The control circuit board 210 may also maintain a data connection (not shown) with the power distribution circuit. The circuit board 210 is also connected to a main controller (not shown) located elsewhere on the lamp head.

A sleeve 202 covers the length of the handle 108 and is attached securely on the handle 108 by release buttons 208.

Operation of the lamp 100 may be considered from a starting point in the off state. A user, say, a surgeon or a nurse, may touch the sensor 204. Indeed, to touch the sensor 204, the user may, for but two examples, use a fingertip or a back of a hand.

Responsive to detecting that the sensor 204 has been touched, the sensor 204 may transmit a signal to the control circuit board 210. Responsive to receiving the signal, the control circuit board 210 may transmit instructions to the main controller. The main controller may transmit instructions to the power distribution circuit. Responsive to receiving the instructions from the main controller and given that the lamp 100 is starting in the off state, the power distribution circuit may simply supply power to the lamp element assemblies 102 and, thereby, to the plurality of lamp elements 104. That is, responsive to the first touch, the lamp 100 is switched to the on state.

Subsequent to switching the lamp 100 to the on state, each touch (tap) may allow the lamp 100 to cycle through different brightness levels. For example, an initial touch may cause the lamp 100 to switch to the on state at 100% brightness. Responsive to receiving a signal indicating that the sensor 204 has detected a subsequent touch, the control circuit board 210 may transmit instructions that act to reduce the power supplied to the lamp element assemblies 102 such that the lamp elements 104 illuminate at an 80% brightness level. Further detected touches may cause the control circuit board 210 may instruct the power distribution circuit to reduce the power supplied to the lamp element assemblies 102 such that the lamp elements 104 illuminate at a 60% brightness level, then a 40% brightness level, then a 20% brightness level and then back to a 100% brightness level.

To return the lamp to the off state, the use may press the sensor 204 and hold the press for a predetermined duration.

Subsequent to switching the lamp 100 to the on state, the user may wish to change the temperature of the light produced by the lamp 100. Temperature in lighting describes how the color of the light appears from a lamp. This temperature is measured in Kelvins (K). 1,000K light is at the “warm” end of the temperature scale and is representative of a light with yellow and red more prominent, like candlelight. 10,000K light is at the “cool” end of the temperature scale and is representative of a light with blue more prominent, like daylight.

It is proposed herein to equip the handle with an axial rotation sensor (not shown) thereby allowing the user to twist the sleeve 202 to cycle through color temperatures.

Responsive to detecting that the sleeve 202 has been twisted, the axial rotation sensor may transmit a signal to the control circuit board 210. Responsive to receiving the signal, the control circuit board 210 may transmit instructions to the main controller. Responsive to receiving the instructions from the control circuit board 210, the main controller may transmit instructions to the lamp element assemblies 102 to customize the operation of the plurality of lamp elements 104 so that the plurality of lamp elements 104 produces light of a different color. For example, the lamp 100 may be turned on with light at 5,000K. A twist of the handle may result in a 500K change in the temperature of the light produced by the plurality of lamp elements 104. Color Temperature may be adjusted in steps of 500K, in a range extending from 3500K to 5000K. The direction of the sensed twist may dictate whether the light temperature is responsively increased or decreased.

As an alternative to the twisting motion, a touch sensor (not shown) located along the sleeve 202 of the handle 108 may be used to detect a sliding motion of a finger lengthwise along the sleeve 202. Responsive to the detection of this sliding motion, the control circuit board 210 may transmit instructions to result in change of color temperature; increasing or decreasing on the basis of the movement direction.

Advantageously, the touch sensor located along the sleeve 202 of the handle 108 may be used to prevent accidental engagement of the touch sensor 204 when the user uses the handle 108 for repositioning the lamp 100. It is preferred to avoid accidentally turning off or changing the brightness of the light. That is, the control circuit board may be adapted to recognize receipt of a signal from the sensor located along the sleeve and, responsive to the receipt, ignore receipt of signals from the sensor 204 for a period of time.

Notably, the sensor 204 may be selected to have a sensitivity sufficient to allow operation in the presence of a sterile disposable cover sheathing the handle 108.

It is contemplated that, through the use of appropriate wired and/or wireless communication protocols, the control circuit board 210 may communicate with power distribution circuits in other lamps (not shown). Accordingly, a user may turn on a plurality of lamps by touching only the sensor 204 at the end of the handle 108 of the lamp 100 of FIG. 1.

The above-described implementations of the present application are intended to be examples only. Alterations, modifications and variations may be effected to the particular implementations by those skilled in the art without departing from the scope of the application, which is defined by the claims appended hereto.

What is claimed is:

1. A lamp comprising:
a lamp element;
a power distribution circuit arranged to control a flow of electricity to the lamp element;
a head housing the lamp element and the power distribution circuit;
a handle attached, at a proximal end, to the head, the handle housing a control circuit board;
a touch sensor in communication with the control circuit board, the sensor disposed at a distal end of the handle;
wherein the control circuit board is adapted to:
receive a signal from the touch sensor, the signal indicative of detection of a touch; and
responsive to the receiving, transmit an instruction to the power distribution circuit, the instruction causing the power distribution circuit to change a state of the lamp element.

2. The lamp of claim 1 wherein the touch sensor comprises a capacitive touch sensor.

3. The lamp of claim 1 further comprising a secondary touch sensor along the handle.

4. The lamp of claim 3 wherein the control circuit board is further adapted to recognize receipt of a signal from the secondary touch sensor and, responsive to the receipt, ignore further receipt of signals from the touch sensor for a period of time.
